(19) United States
(12) Patent Application Publication
(10) Pub. No.: US 2023/0377734 A1
(43) Pub. Date: Nov. 23, 2023

(54) MEDICAL INFORMATION PROCESSING DEVICE AND MEDICAL INFORMATION PROCESSING METHOD

(71) Applicant: CANON MEDICAL SYSTEMS CORPORATION, Otawara-shi (JP)

(72) Inventor: Shiro KATO, Utsunomiya (JP)

(73) Assignee: CANON MEDICAL SYSTEMS CORPORATION, Otawara-shi (JP)

(21) Appl. No.: 18/317,450

(22) Filed: May 15, 2023

(30) Foreign Application Priority Data
May 18, 2022 (JP) 2022-081435

(51) Int. Cl. G16H 40/40

(52) U.S. Cl. CPC G16H 40/40

(57) ABSTRACT

A medical information processing device of an embodiment includes processing circuitry. The processing circuitry is configured to acquire first information regarding medical data and second information regarding an analysis application used to analyze the medical data, and determine a possibility of failure of analysis of the medical data by an analysis application selected from a plurality of analysis applications on the basis of the first information and the second information.

### CROSS-REFERENCE TO RELATED APPLICATION

The present application claims priority based on Japanese Patent Application No. 2022-081435 filed May 18, 2022, the content of which is incorporated herein by reference.

### FIELD

Embodiments disclosed in the present specification and drawings relate to a medical information processing device and a medical information processing method.

### BACKGROUND

Conventionally, there are automatic analysis devices that are used by being directly connected to modalities. An automatic analysis device directly receives medical images generated by a modality and automatically selects an appropriate analysis application on the basis of supplementary information and pixel data of the received medical images. The automatic analysis device activates the selected analysis application and outputs or transmits the results.

In conventional automatic analysis devices, as a result of referring to analysis results of an analysis application, there may be defects in analysis results, such as analysis failures and many misdetections. However, defects are discovered after analysis is actually performed using an analysis application in many cases, and thus it is difficult to predict defects.

### BRIEF DESCRIPTION OF THE DRAWINGS

FIG. 1 is a diagram showing an example of a usage environment and functional blocks of a medical information processing system 1 according to an embodiment.

FIG. 2 is a diagram showing an example of functional blocks of an automatic analysis device 20.

FIG. 3 is a diagram showing an example of functional blocks of a medical information processing device 100.

FIG. 4 is a diagram showing an example of the contents of a determination result DB 151.

FIG. 5 is a flowchart showing an example of processing in the medical information processing device 100.

FIG. 6 is a diagram showing an overview of a processing flow before analysis by the automatic analysis device 20 is started.

FIG. 7 is a diagram showing an example of an image showing failure suggestion information displayed on a display.

FIG. 8 is a diagram showing an example of processing of the automatic analysis device 20.

FIG. 9 is a flowchart showing an example of processing in the medical information processing device 100.

FIG. 10 is a diagram showing an overview of a processing flow after analysis by the automatic analysis device 20 is started.

### DETAILED DESCRIPTION

A medical information processing device and a medical information processing method will be described below with reference to the drawings.

A medical information processing device of an embodiment includes processing circuitry. The processing circuitry is configured to acquire first information regarding medical data and second information regarding an analysis application used to analyze the medical data, and determine a possibility of failure of analysis of the medical data by an analysis application selected from a plurality of analysis applications on the basis of the first information and the second information.

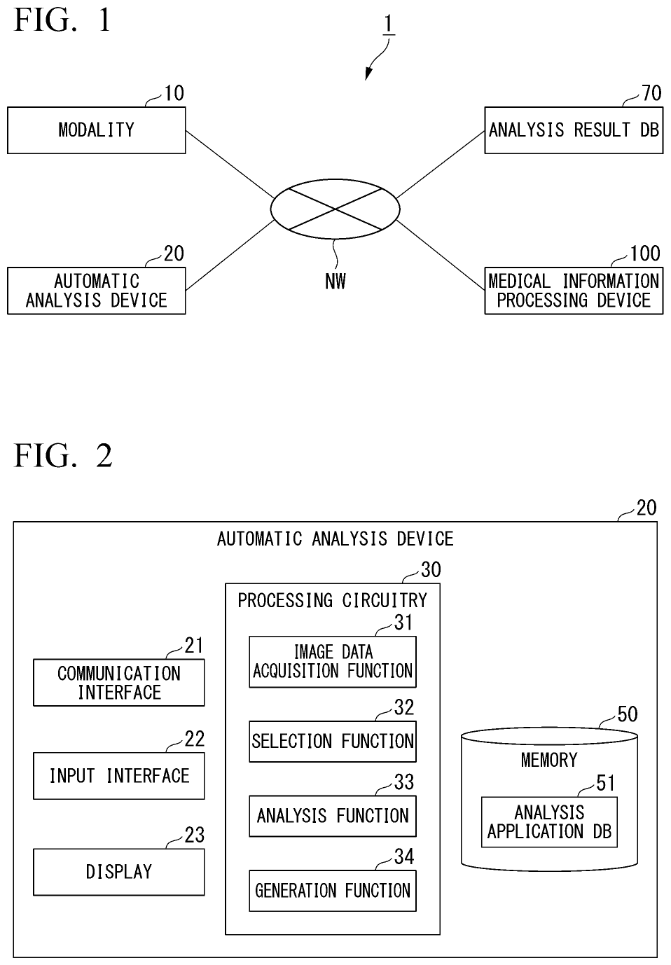

FIG. 1 is a diagram showing an example of a usage environment and functional blocks of a medical information processing system 1 according to an embodiment. The medical information processing system 1 includes, for example, a modality 10, an automatic analysis device 20, an analysis result database (hereinafter, DB) 70, and a medical information processing device 100. The modality 10, the automatic analysis device 20, the analysis result DB 70, and the medical information processing device 100 are connected such that they can communicate via a network NW.

The modality 10 performs photographing (imaging) according to imaging conditions (imaging protocol) determined on the basis of an imaging examination order, for example. Examples of the modality 10 may include an X-ray computed tomography apparatus, an X-ray diagnostic apparatus, a magnetic resonance imaging apparatus, an ultrasonic diagnostic apparatus, a nuclear medicine diagnostic apparatus, and the like. The modality 10 is operated, for example, by a doctor such as a radiologist or an operator such as a radiographer. The modality 10 transmits a medical image (medical image data) generated by imaging to the automatic analysis device 20 or an external apparatus such as a picture archiving and communication system (PACS) which is not shown.

The automatic analysis device 20 automatically selects an analysis application (hereinafter, analysis application) suitable for analyzing medical image data by conditionally branching information obtained from supplementary information and pixel data of medical image data transmitted by the modality 10, for example. The automatic analysis device 20 analyzes medical image data using the selected analysis application. The automatic analysis device 20 may be directly connected to the modality 10 without the network NW.

FIG. 2 is a diagram showing an example of functional blocks of the automatic analysis device 20. The automatic analysis device 20 includes, for example, a communication interface 21, an input interface 22, a display 23, processing circuitry 30, and a memory 50.

The communication interface 21 communicates with external devices such as the modality 10, the analysis result DB 70, and the medical information processing device 100, for example, via a network NW such as a LAN. The communication interface 21 includes, for example, a communication interface such as a network interface card (NIC). The network NW may include the Internet, a cellular network, a Wi-Fi network, a wide area network (WAN), and the like instead of or in addition to the LAN.

The input interface 22 receives various input operations from an operator. The input interface 22 converts received input operations into electrical signals and transmits the electrical signals to the processing circuitry 30. The input interface 22 generates information according to an input operation when the operator performs the input operation. The input interface 22 transmits the generated information according to the input operation to the processing circuitry 30.

The input interface 22 includes, for example, a mouse, a keyboard, a trackball, a switch, a button, a joystick, a touch panel, and the like. The input interface 22 may be, for example, a user interface that receives voice input, such as a microphone. When the input interface 22 is a touch panel, the input interface 22 may also have a display function of the display 23.

The input interface in this specification is not limited to those having physical operation parts such as a mouse and a keyboard. For example, examples of the input interface also include electrical signal processing circuitry that receives an electrical signal corresponding to an input operation from an external input apparatus provided separately from the device and outputs the electrical signal to a control circuit.

The display 23 displays various types of information. For example, the display 23 displays an image generated by processing circuitry 140, a graphical user interface (GUI) for receiving various input operations from the operator, and the like. For example, the display 23 is a liquid crystal display (LCD), a cathode ray tube (CRT) display, an organic electroluminescence (EL) display, or the like.

The processing circuitry 30 includes, for example, an image data acquisition function 31, a selection function 32, an analysis function 33, and a generation function 34. The processing circuitry 30 realizes these functions by a hardware processor (computer) executing a program stored in a memory (storage circuit) 50, for example.

The hardware processor means, for example, circuitry such as a CPU, a graphics processing unit (GPU), an application specific integrated circuit (ASIC), and a programmable logic device (e.g., a simple programmable logic device (SPLD), a complex programmable logic device (CPLD), and a field programmable gate array (FPGA)).

Instead of storing the program in the memory 50, the program may be directly incorporated into the circuit of the hardware processor. In this case, the hardware processor realizes the functions by reading and executing the program incorporated into the circuit. The aforementioned program may be stored in a memory 150 in advance, or may be stored in a non-transitory storage medium such as a DVD or CD-ROM and installed into memory 50 from the non-transitory storage medium when the non-transitory storage medium is set in a drive device (not shown) of the automatic analysis device 20.

The hardware processor is not limited to being configured as a single circuit, and may be configured as a single hardware processor by combining a plurality of independent circuits to realize each function. Further, a plurality of components may be integrated into one hardware processor to realize each function. The memory 50 stores an analysis application DB 51. The analysis application DB 51 includes a plurality of types of analysis applications used for medical image data analysis processing. An application activation rule is set for each analysis application.

The image data acquisition function 31 in the processing circuitry 30 acquires medical image data transmitted by the modality 10. For example, supplementary information is added to the medical image data. The supplementary information includes, for example, contrast information including information on whether a contrast medium is used at the time of imaging, and information such as reconstruction functions (reconstruction function conditions) at the time of reconstructing medical image data.

The image data acquisition function 31 transmits medical image data information related to acquired medical image data to the medical information processing device 100 along with supplementary information. Medical image data is an example of medical data. Medical image data information is an example of first information. The first information may include information other than medical image data information, or may be information other than medical image data information related to medical image data.

The selection function 32 selects an analysis application suitable for analyzing medical image data from a plurality of analysis applications included in the analysis application DB 51 on the basis of supplementary information added to medical image data and pixel data of the medical image data. At the time of selecting an analysis application, the selection function 32 refers to an application activation rule set for analysis applications to select an analysis application according to the application activation rule.

The selection function 32 transmits selected analysis application information regarding the selected analysis application to the medical information processing device 100. The selection function 32 adds the medical image data to the selected analysis application information. The selected analysis application information includes an application activation rule. The selected analysis application information is an example of second information regarding an analysis application used for analyzing medical image data. The second information may include information other than the selected analysis application information, or may be information other than the selected analysis application information related to the selected analysis application information.

The selection function 32 modifies the application activation rule on the basis of activation rule correction information transmitted by the medical information processing device 100, which will be described later, and stores it in the analysis application DB 51. When the application activation rule has been modified, the selection function 32 selects an analysis application according to the modified application activation rule.

The analysis function 33 analyzes medical image data acquired by the image data acquisition function 31 using the analysis application selected by the selection function 32 and generates analysis data. The analysis function 33 determines whether or not the analysis application has been activated at the time of generating the analysis data. The analysis function 33 generates analysis result data including data indicating that analysis is successful (hereinafter, successful analysis data) when the analysis application is activated and the analysis data is successfully generated, and generates analysis result data including data indicating that analysis has failed (hereinafter, failed analysis data) when the analysis application is not activated and analysis has failed. Analysis result data includes not only success or failure of analysis but also supplementary information of the analyzed medical image data and information indicating the analysis application used for analysis.

The analysis function 33 causes the display 23 to display medical image data and analysis data generated by analyzing the medical image data. The analysis function 33 transmits the generated analysis result data to other external apparatuses such as the medical information processing device 100 and the analysis result DB 70, or causes the display 23 to display the analysis result data. The analysis function 33 may cause the display 23 to display analysis data if the analysis result data includes successful analysis data and causes the display 23 to display failure of analysis if the analysis result data includes failed analysis data.

The display 23 displays analysis data generated by the analysis function 33, for example. An operator such as a radiologist determines whether the analysis data displayed on the display 23 is successfully analyzed data or failed data. The radiologist determines whether the analysis data is successfully analyzed data or failed data according to whether or not the generated analysis data has many misdetections. The operator inputs results of his own determination to the input interface 22.

The input interface 22 transmits success identification information to the processing circuitry 30 when information indicating that the analysis data is successfully analyzed data is input. The input interface 22 transmits failure identification information to the processing circuitry 30 when information indicating that the analysis data is data that has failed to be analyzed is input. The analysis function 33 includes, in success analysis data, the success identification information or the failure identification information transmitted by the input interface 22. The success identification information and the failure identification information may be generated using, for example, a trained model generated by machine learning using medical image data as input data and using output data indicating whether an analysis result is success or failure.

The generation function 34 generates request information for requesting that the medical information processing device 100 determine whether an analysis application to be used is suitable for analysis when the automatic analysis device 20 analyzes medical image data, and transmits the request information to the medical information processing device 100. The request information includes medical image data to be analyzed, supplementary information thereof, and analysis application identification information for identifying an analysis application to be used for analysis.

The analysis result DB 70 stores analysis result data generated by analyzing medical image data by the automatic analysis device 20 in the past. Analysis result data stored in the analysis result DB 70 includes successful analysis data and failed analysis data. The successful analysis data includes along with either success identification information or failure identification information, and reconstruction conditions, for example, reconstruction function conditions, of the modality 10 that has acquired the medical image data for which the analysis result data has been generated.

The analysis result DB 70 is data collected by associating application activation rules, supplementary information of medical image data, analysis result data, success identification information, and failure identification information. The analysis result DB 70 provides stored analysis results to the medical information processing device 100 in response to a request from the medical information processing device 100. The analysis result DB 70 may store analysis data along with analysis result data. The analysis result DB 70 may be included in the automatic analysis device 20.

The medical information processing device 100 determines success or failure of analysis on the basis of supplementary information, an application activation rule, or analysis result data transmitted by the automatic analysis device 20. The medical information processing device 100 transmits various types of information to the modality 10 and the automatic analysis device 20 according to the determined success or failure of analysis.

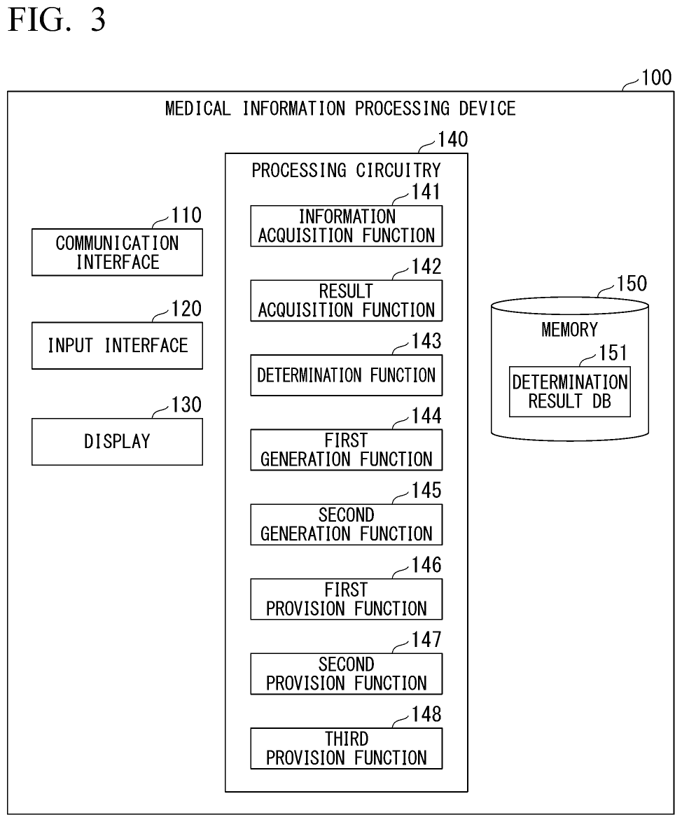

FIG. 3 is a diagram showing an example of functional blocks of the medical information processing device 100. The medical information processing device 100 includes, for example, a communication interface 110, an input interface 120, a display 130, processing circuitry 140, and a memory 150.

The communication interface 110 communicates with external devices such as the modality 10 and the automatic analysis device 20, for example, via a network NW such as a LAN. The communication interface 110 includes, for example, a communication interface such as an MC. The network NW may include the Internet, a cellular network, a Wi-Fi network, a WAN, and the like instead of or in addition to the LAN.

The input interface 120 includes, for example, a mouse, a keyboard, a trackball, a switch, a button, a joystick, a touch panel, and the like. The input interface 120 may be, for example, a user interface that receives voice input, such as a microphone. If the input interface 120 is a touch panel, the input interface 120 also has the display function of the display 130.

The display 130 displays various types of information. For example, the display 130 displays an image generated by the processing circuitry 140, a GUI for receiving various input operations from the operator, and the like. For example, the display 23 is an LCD, a CRT display, an organic EL display, or the like.

The processing circuitry 140 includes, for example, an information acquisition function 141, a result acquisition function 142, a determination function 143, a first generation function 144, a second generation function 145, a first provision function 146, a second provision function 147, and a third provision function 148. The processing circuitry 140 realizes these functions by a hardware processor executing a program stored in the memory 150, for example.

Instead of storing the program in the memory 150, the program may be directly incorporated into the circuit of the hardware processor. In this case, the hardware processor realizes the functions by reading and executing the program incorporated into the circuit. The aforementioned program may be stored in the memory 150 in advance, or may be stored in a non-transitory storage medium such as a DVD or a CD-ROM and installed into the memory 150 from the non-transitory storage medium when the non-transitory storage medium is set in a drive device (not shown) of the medical information processing device 100.

The hardware processor is not limited to being configured as a single circuit, and may be configured as one hardware processor by combining a plurality of independent circuits to realize each function. Further, a plurality of components may be integrated into one hardware processor to realize each function. The memory 150 stores a determination result DB 151.

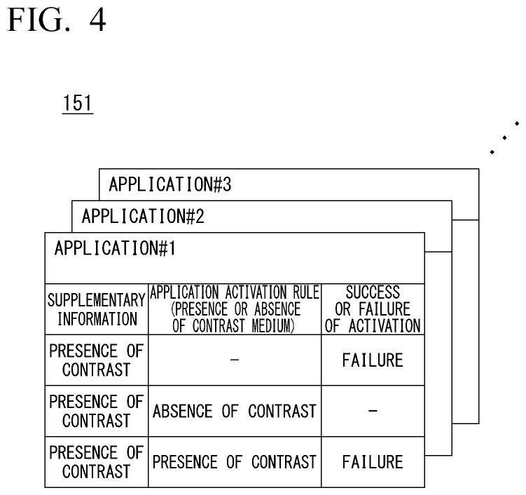

FIG. 4 is a diagram showing an example of the contents of the determination result DB 151. The determination result DB 151 includes supplementary information in a plurality of analysis applications and results of success or failure of activation when an application activation rule has been set. For example, in an analysis application “application #1,” at the time of analyzing medical image data that satisfies the condition that “contrast information” included in “supplementary information” is “with contrast,” activation of the analysis application fails in the case where contrast information is “with contrast” “-” (no setting regarding presence or absence of a contrast medium) as an application activation rule, and the analysis application is successfully activated in the case of “no contrast” as an application activation rule. The determination result DB 151 includes similar determination results for “application #2,” “application #3,” and the like.

The information acquisition function 141 in the processing circuitry 140 acquires medical image data information, selected analysis application information, and request information transmitted by the automatic analysis device 20. The information acquisition function 141 is an example of an information acquisition unit. The information acquisition function 141 acquires analysis result data information transmitted by the input interface 120. The information acquisition function 141 acquires success identification information and failure identification information when successful analysis data is included in the acquired analysis result data information.

The result acquisition function 142 acquires analysis result data information transmitted by the automatic analysis device 20. The result acquisition function 142 acquires success or failure of generation of analysis data as correctness or incorrectness of an analysis result using a selected analysis application during analysis processing depending on whether the acquired analysis result data information includes any of successful analysis data and failed analysis data. The result acquisition function 142 acquires incorrectness of the used analysis result if the acquired analysis result data information includes failed analysis data. The result acquisition function 142 is an example of a result acquisition unit.

If the analysis result data information includes successful analysis data, the result acquisition function 142 acquires determination of success or failure of analysis of the analysis data by the radiologist as correctness or incorrectness of the analysis result using the selected analysis application during analysis processing depending on whether the successful analysis data includes any of the success identification information and the failure identification information.

The determination function 143 determines whether a possibility of failure of analysis of medical image data by the analysis application (hereinafter, possibility of failure) is high on the basis of information acquired by the information acquisition function 141. For example, the determination function 143 searches the analysis result DB 70 for analysis result data acquired by the result acquisition function 142, supplementary information included in request information, and analysis result data shared by analysis applications.

The determination function 143 determines that the possibility of failure is high when medical image data and the analysis application included in the analysis result data information and the request information have been used on the basis of information on success or failure of analysis included in the analysis result data searched from the analysis result DB 70. The determination function 143 may calculate a possibility of failure as a numerical value using a predetermined arithmetic expression or the like, and determine that the possibility of failure is high when the possibility of failure exceeds a threshold value. The determination function 143 is an example of a determination unit.

The determination function 143 determines that the possibility of failure is high when the medical image data and the analysis application included in the analysis result data information have been used in a case where the analysis result data including the successful analysis data includes the failure identification information. The case where the analysis result data including the successful analysis data includes the failure identification information is a case where the radiologist who has viewed analysis data determines that the analysis data is data that has failed to be analyzed.

The first generation function 144 generates change information for changing reconstruction function conditions for reconstructing medical image data in the modality 10 when the determination function 143 determines that the radiologist determines analysis data to be data that has failed to be analyzed. For example, the first generation function 144 reads the determination result DB 151 stored in the memory 150, classifies analysis result data for each reconstruction function condition, and selects analysis result data of reconstruction function conditions with few examples to which failure identification information has been added. The first generation function 144 generates change information for changing the reconstruction function conditions to the reconstruction function conditions in the selected analysis result data. A reconstruction condition (reconstruction function condition) is an example of a construction condition for constructing medical data.

The construction condition may be other conditions. The construction condition may be, for example, an imaging condition (imaging protocol) for imaging a subject in the modality 10 or a processing condition for image processing of medical image data in the automatic analysis device 20. The first generation function 144 is an example of a first generation unit.

When the result acquisition function 142 acquires analysis result data information including failed analysis data and the determination function 143 determines that the possibility of failure is high, the second generation function 145 reads the determination result DB 151 stored in the memory 150 and generates activation rule correction information for changing an application activation rule in an analysis application according to an example of successful activation. The second generation function 145 may search for an analysis application that has been successfully activated as a result of referring to supplementary information and the application activation rule, and generate the activation rule correction information using the discovered analysis application. The second generation function 145 is an example of a second generation unit.

If the determination function 143 determines that the possibility of failure is low when the medical image data and the analysis application included in the analysis result data information have been used, the first provision function 146 generates success suggestion information indicating that the possibility of failure is low. If the determination function 143 determines that the possibility of failure is high when the medical image data and the analysis application included in the analysis result data information have been used, the first provision function 146 generates failure suggestion information indicating that the possibility of failure is high. The first provision function 146 provides the generated success suggestion or failure suggestion information by causing the display 130 to display it or transmitting it to an external device such as the modality 10 or the automatic analysis device 20. The first provision function 146 is an example of a first provision unit. The first provision function 146 also serves as an output unit, for example.

The second provision function 147 provides the change information generated by the first generation function 144 by transmitting it to the modality 10. The second provision function 147 may cause the display 130 to display the reconstruction function conditions changed on the basis of the transmitted change information or transmit them to an external device other than the modality 10. The second provision function 147 is an example of a second provision unit. The second provision function 147 also serves as an output unit, for example.

The third provision function 148 provides activation rule correction information generated by the second generation function 145 by transmitting it to the automatic analysis device 20. The third provision function 148 may provide the activation rule correction information by causing the display 130 to display it or transmit it to an external device other than the automatic analysis device 20. The third provision function 148 is an example of a third provision unit. The third provision function 148 also serves as an output unit, for example.

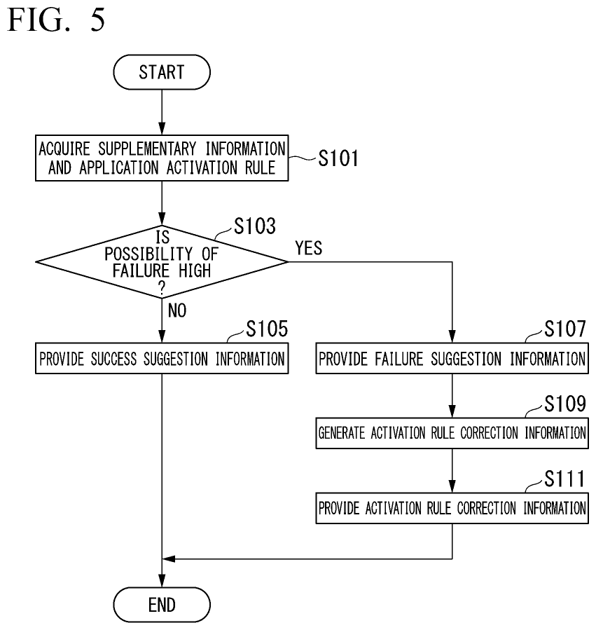

Next, processing in the medical information processing device 100 will be described. In the medical information processing device 100, processing before analysis by the automatic analysis device 20 is started and processing after analysis by the automatic analysis device 20 is started are executed. FIG. 5 is a flowchart showing an example of processing in the medical information processing device 100. FIG. 5 shows processing of the medical information processing device 100 before analysis by the automatic analysis device 20 is started.

Prior to processing performed by the medical information processing device 100, if the automatic analysis device 20 acquires medical image data to be analyzed through the modality 10, the selection function 32 selects an analysis application to be used for analyzing medical image data information. Subsequently, the generation function 34 generates request information with reference to the medical image data information and the analysis application and transmits the request information to the medical information processing device 100. Thereafter, processing in the medical information processing device 100 is started.

The medical information processing device 100 to which the request information has been transmitted acquires supplementary information of the medical image data and an application activation rule of the analysis application included in the request information in the information acquisition function 141 (step S101). Subsequently, the determination function 143 determines whether or not a possibility of failure of analysis of the medical image data by the analysis application acquired by the information acquisition function 141 is high with reference to analysis result data stored in the analysis result DB 70 (step S103). If the determination function 143 determines that the possibility of failure is not high (the possibility of failure is low), the first provision function 146 generates success suggestion information. The first provision function 146 provides the success suggestion information by transmitting it to an external apparatus or causing the display 130 to display it (step S105). Thereafter, the medical information processing device 100 ends processing shown in FIG. 5.

The first provision function 146 generates failure suggestion information if the determination function 143 determines that the possibility of failure is high. The first provision function 146 provides the failure suggestion information by transmitting it to an external apparatus or causing the display 130 to display it (step S107). Subsequently, when the result acquisition function 142 has acquired analysis result data information including failed analysis data, the second generation function 145 reads the determination result DB 151 stored in the memory 150 and generates activation rule correction information for changing the application activation rule in the analysis application according to an example of successful activation (step S109).

Subsequently, the third provision function 148 provides the activation rule correction information generated by the second generation function 145 by transmitting it to the automatic analysis device 20 (step S111). In the automatic analysis device 20, the selection function 32 corrects the application activation rule according to the transmitted activation rule correction information, and then re-selects an analysis application. In this manner, the medical information processing device 100 ends processing of the flow shown in FIG. 5.

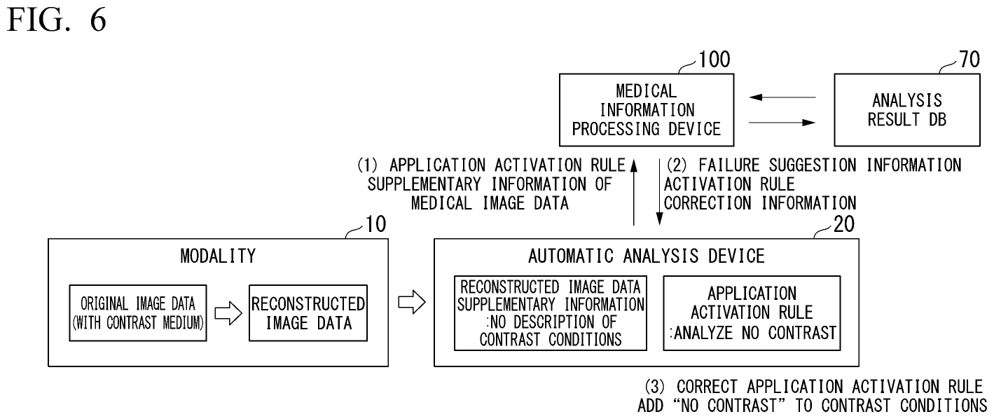

Next, an example of a processing flow between devices before analysis by the automatic analysis device 20 is started will be described. FIG. 6 is a diagram showing an overview of a processing flow before analysis by the automatic analysis device 20 is started. The modality 10 acquires original image data by imaging a subject and generates reconstructed image data by reconstructing the original image data. In this example, a contrast medium has been administered to the subject, and the original image data is image data with the contrast medium. The modality 10 transmits the generated reconstructed image data to the automatic analysis device 20 as medical image data. It is assumed that there is no description of, for example, “contrast conditions” as contrast information included in supplementary information attached to the medical image data. The original image data is an example of original medical image data. The reconstructed medical image data is an example of reconstructed image data.

The automatic analysis device 20 selects an analysis application with reference to an application activation rule of “no contrast” on the basis of the reconstructed image data (medical image data) transmitted by the modality 10. The automatic analysis device 20 transmits, to the medical information processing device 100, request information including medical image data information having no description of contrast conditions as contrast information and selected analysis application information indicating selection of an analysis application with the application activation rule of “no contrast.”

The medical information processing device 100 searches the analysis result DB 70 for supplementary information included in the transmitted request information and analysis result data sharing the application activation rule and determines whether there is a high possibility of failure. If the medical information processing device 100 determines that the possibility of failure is high, it generates failure suggestion information and activation rule correction information and transmits them to the automatic analysis device 20.

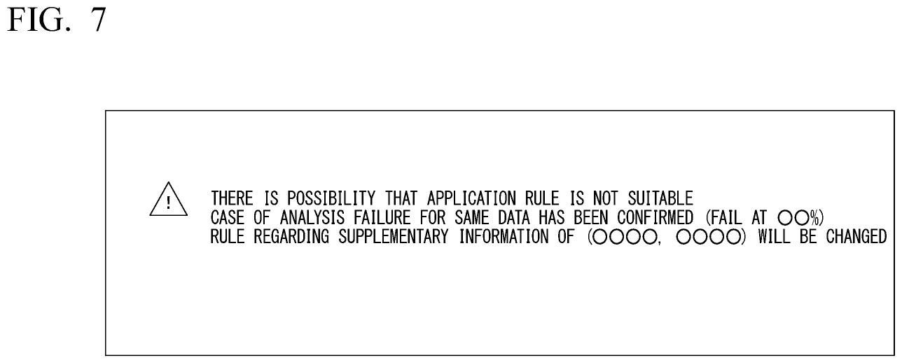

The automatic analysis device 20 causes the display 23 to display the transmitted failure suggestion information. FIG. 7 is a diagram showing an example of an image showing failure suggestion information displayed on the display. In the example shown in FIG. 7, the medical information processing device 100 calculates a possibility of failure, and it is displayed as (failure at ◯◯%) on an image of the display 23. Furthermore, the fact that the activation rule will be corrected on the basis of the activation rule correction information, and the activation rule to be corrected are displayed.

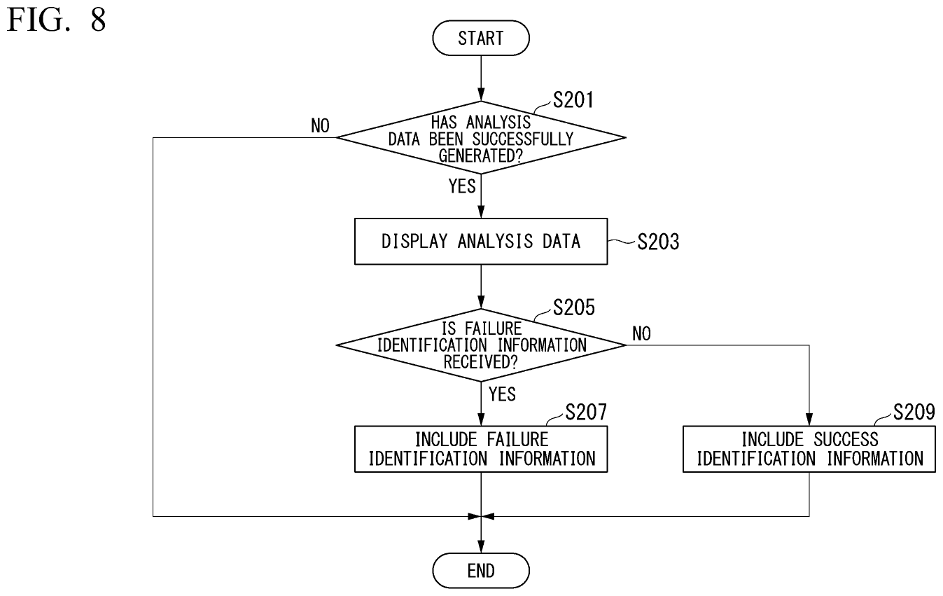

Next, processing after analysis by the automatic analysis device 20 is started will be described, but before that, a part of processing of the automatic analysis device 20 after analysis by the automatic analysis device 20 is started will be described. FIG. 8 is a diagram showing an example of processing of the automatic analysis device 20. After receiving medical image data transmitted by the modality 10, the automatic analysis device 20 selects an analysis application using the selection function 32 and analyzes the medical image data using the analysis function 33.

Subsequently, the analysis function 33 determines whether or not analysis data has been successfully generated (step S201). If the analysis function 33 determines that the analysis data has not been successfully generated, the automatic analysis device 20 ends processing shown in FIG. 8 as it is. In this case, the automatic analysis device 20 generates analysis result data information including failed analysis data.

If it is determined that the analysis data has been successfully generated, the analysis function 33 causes the display 23 to display the generated analysis data (step S203). The analysis data displayed on the display 23 can be seen by, for example, a radiologist. The radiologist views the analysis data displayed on the display 23, determines success or failure of analysis, for example, according to whether or not the generated analysis data is successful enough to be used for radiogram interpretation, and operates the input interface 22 on the basis of results of own determination.

The input interface 22 transmits success identification information when information indicating that analysis is successful is input and transmits failure identification information when information indicating that analysis has failed is input. The analysis function 33 determines whether the failure identification information has been received (success identification information has been received) (step S205).

When the failure identification information has been received, the analysis function 33 includes the failure identification information in successful analysis data (step S207). When the failure identification information has not been received (success identification information has been received), the analysis function 33 includes the success identification information in the successful analysis data (step S209). Thus, the automatic analysis device 20 ends processing shown in FIG. 8.

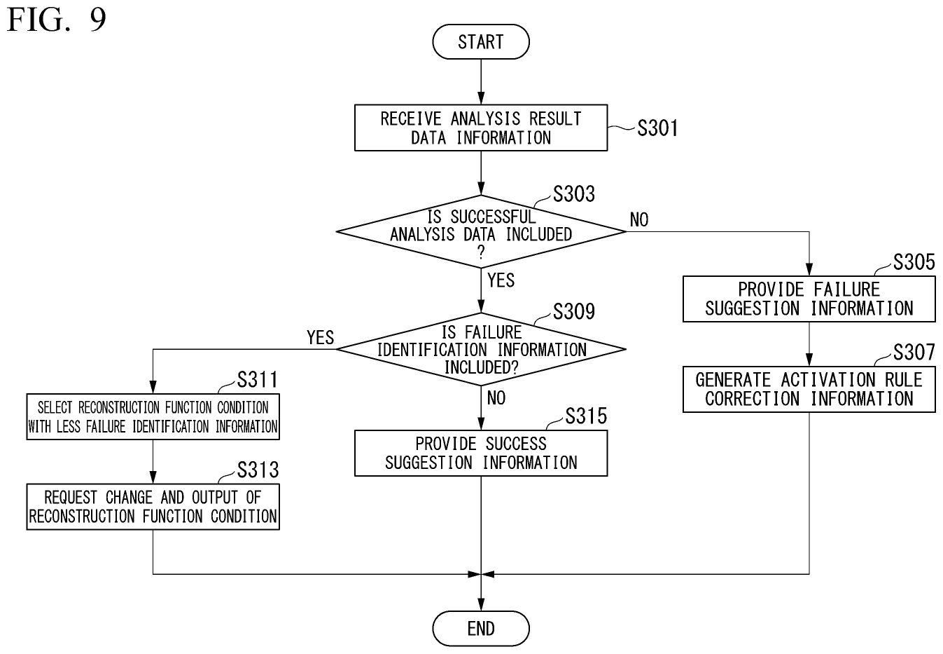

Next, processing in the medical information processing device 100 after analysis by the automatic analysis device 20 is started will be described. FIG. 9 is a flowchart showing an example of processing in the medical information processing device 100. FIG. 9 shows processing of the medical information processing device 100 before analysis by the automatic analysis device 20 is started.

First, the medical information processing device 100 receives and acquires analysis result data information transmitted by the automatic analysis device 20 using the information acquisition function 141 (step S301). Subsequently, the information acquisition function 141 determines whether or not the acquired analysis result data information includes successful analysis data (step S303).

If it is determined that the acquired analysis result data information includes failed analysis data instead of the successful analysis data, the first provision function 146 transmits failure suggestion information to an external apparatus or causes the display 130 to display it (step S305). Subsequently, when the result acquisition function 142 acquires analysis result data information including failed analysis data, the second generation function 145 reads the determination result DB 151 stored in the memory 150, generates activation rule correction information for changing the application activation rule in the analysis application depending on an example of successful activation (step S307), and transmits the activation rule correction information to the automatic analysis device 20.

If it is determined that the acquired analysis result data information includes successful analysis data, the result acquisition function 142 determines whether or not the successful analysis data includes failure identification information (step S309). If it is determined that the successful analysis data includes the failure identification information, the determination function 143 determines that a radiologist has determined that analysis data is data that has failed in medical image data analysis.

In this case, the first generation function 144 selects a reconstruction function condition with less failure identification information (step S311) and generates change information for changing a reconstruction function condition of the modality 10 to the selected reconstruction function condition. Subsequently, the second provision function 147 transmits the change information generated by the first generation function 144 to the modality 10 and sends an output request to the modality 10 such that the reconstruction function condition is changed and reconstructed image data is generated again in the changed reconstruction function condition and transmitted to the automatic analysis device 20 (step S313). Thereafter, the medical information processing device 100 ends processing shown in FIG. 9.

When the result acquisition function 142 determines that the successful analysis data includes the success identification information instead of the failure identification information in step S309, the first provision function 146 generates success suggestion information and transmits it to the automatic analysis device 20 (step S315). Thereafter, the medical information processing device 100 ends processing shown in FIG. 9.

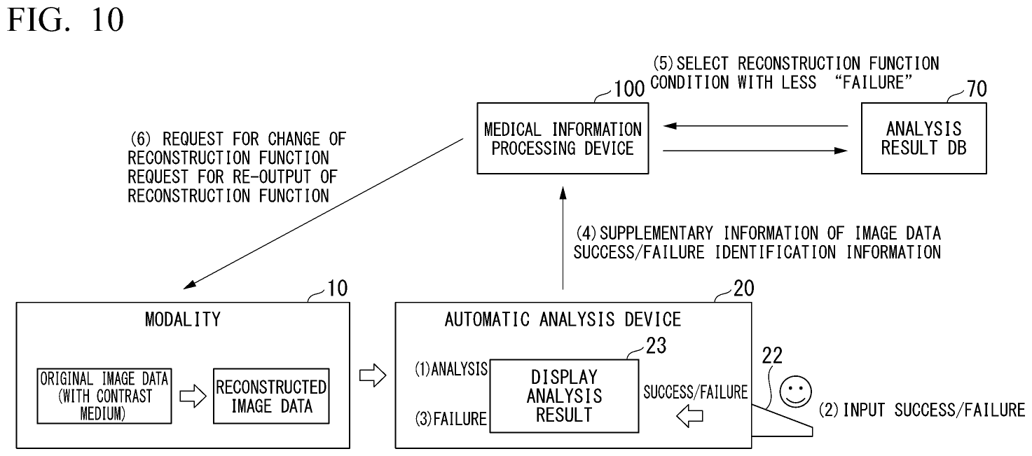

Next, an example of a processing flow between devices after analysis by the automatic analysis device 20 is started will be described. FIG. 10 is a diagram showing an overview of a processing flow after analysis by the automatic analysis device 20 is started. The modality 10 acquires original image data by imaging a subject and generates reconstructed image data by reconstructing the original image data. In this example, a contrast medium has been administered to the subject, and the original image data is image data to which contrast information indicating “presence of contrast medium” has been added.

The modality 10 images the subject to acquire original image data and generates reconstructed image data by reconstructing the original image data. In this example, a contrast medium has been administered to the subject, and the original image data is image data to which contrast information indicating “presence of contrast medium” has been added. The modality 10 transmits the generated reconstructed image data to the automatic analysis device 20 as medical image data.

The automatic analysis device 20 selects an analysis application for analyzing the reconstructed image data (medical image data) transmitted by the modality 10 using the selection function 32 and analyzes the reconstructed image data. Subsequently, the display 23 displays analysis data generated by analyzing the reconstructed image data using the selected application.

A radiologist who views the analysis data displayed on the display 23 determines whether the analysis data is data that has succeeded or failed in analysis, and inputs a determination result into the input interface 22. In the example shown in FIG. 10, the radiologist inputs a result of determination that the analysis data is data that has failed in analysis.

Subsequently, the automatic analysis device 20 transmits, to the medical information processing device 100, supplementary information of image data (medical image data) included in the medical image data information, and success identification information or failure identification information (here, failure identification information) according to determination of the radiologist. The medical information processing device 100 selects a reconstruction function condition with less analysis result data including failure identification information from analysis result data sharing the transmitted supplementary information.

The medical information processing device 100 requests change of the reconstruction function condition by transmitting change information for changing the reconstruction function condition to the selected reconstruction function condition to the modality 10. Furthermore, the medical information processing device 100 reconstructs and generates the original image data using the changed reconstruction function and requests re-output of the generated reconstructed image data to the automatic analysis device 20.

The medical information processing device 100 according to the embodiment determines whether or not a possibility of failure of activation of an analysis application is high at the time of analyzing medical image data using the analysis application on the basis of supplementary information of the medical image data and an application activation rule of the analysis application. Therefore, it is possible to predict defects in analysis results.

Conventionally, for example, as a result of analysis of medical image data by the automatic analysis device 20 using an analysis application after acquiring the medical image data, the analysis may fail or misdetection may occur many times. In such a case, it is difficult to ascertain whether analysis failure or misdetection (hereinafter, failure or the like) is caused by the application activation rule of the automatic analysis device 20, medical image data (reconstructed image data) generated by the modality 10, or characteristics of the analysis application itself at the time of analyzing medical image data. Therefore, if analysis fails or misdetection occurs many times, it takes a long time for re-analysis.

In this respect, by using the medical information processing device 100 of the embodiment, it is possible to easily identify which of an application activation rule, medical image data (reconstructed image data), and an analysis application causes a failure. Furthermore, since the relationship between supplementary information of medical image data that fails to activate the application and the application activation rule can be acquired, it is possible to reduce a time required for re-analysis when the failure or the like is caused by the application activation rule.

Further, even if the failure or the like is caused by the medical image data, medical image data can be generated in a reconstruction function condition suitable for analysis of the application because the reconstruction function condition of the medical image data can be changed. Further, if there is a problem in the characteristics of the analysis application itself at the time of analyzing the medical image data, the analysis application can be updated and further analysis can be performed using another analysis application until update is completed. Therefore, it is possible to reduce a time required for re-analysis when the failure or the like is caused by the medical image data or the analysis application itself.

Further, information such as failure suggestion information and success suggestion information (hereinafter, provision information) may be provided in a manner other than being displayed on the display 23 of the automatic analysis device 20. For example, the provision information may be displayed on the console (display unit) of the modality 10 or may be provided by being transmitted to a terminal device carried by a doctor or the like through e-mail. Further, medical image data may be displayed in a study list of a PACS to which it is transmitted, or may be displayed on the mobile viewer in the same manner as the PACS. Alternatively, the provision information may be provided by being written to a log of an application such as an analysis application. A mode of provision may be, for example, output as a dialog, embedded input for analysis result data, transmission of e-mail, recording in a log, transmission in a text format using http communication, or the like.

In addition, the provision information may include information other than the information shown in the above-described embodiment. At the time of proposing an analysis application to be selected, the provision information may include, for example, a failure rate (success rate) when analysis has failed, the reason for a failure, a correction (change) part when correction (change) is performed, notification of an improved part at the time of performing similar analysis next time, and the like. Alternatively, when medical image data has been analyzed using an analysis application, the provision information may include details of correction and the like, a failure rate (success rate) when analysis has failed, the reason for a failure, a correction (change) part when correction (change) has been performed, and the like.

The provision information may be provided at any timing. The provision information may be provided, for example, before execution of analysis by an analysis application, for example, before selection of an analysis application using an application activation rule, or immediately before the analysis application is activated. Alternatively, the provision information may be provided, for example, after analysis is performed by the analysis application, for example, immediately after analysis results are transmitted or after a radiologist performs an input operation for generating success identification information or failure identification information.

According to at least one embodiment described above, it is possible to predict defects in analysis results by including an information acquisition unit configured to acquire first information regarding medical data and second information regarding an analysis application used to analyze the medical data, and a determination unit configured to determine a possibility of failure of analysis of the medical data by an analysis application selected from a plurality of analysis applications on the basis of the first information and the second information.

The embodiment described above can be represented as follows.

A medical information processing device including processing circuitry,

wherein the processing circuitry acquires first information regarding medical data and second information regarding an analysis application used to analyze the medical data, and determines a possibility of failure of analysis of the medical data by an analysis application selected from a plurality of analysis applications on the basis of the first information and the second information.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical information processing device comprising processing circuitry configured to:
acquire first information regarding medical data and second information regarding an analysis application used to analyze the medical data; and
determine a possibility of failure of analysis of the medical data by an analysis application selected from a plurality of analysis applications on the basis of the first information and the second information.

2. The medical information processing device according to claim 1, wherein the processing circuitry is further configured to provide failure suggestion information indicating that the possibility is high upon determining that the possibility is high.

3. The medical information processing device according to claim 1, wherein the processing circuitry is further configured to:
generate correction information for correcting construction conditions for constructing medical data upon determining that the possibility is high; and
provide the correction information.

4. The medical information processing device according to claim 1, wherein
the medical data includes medical image data, and
the first information includes supplementary information of the medical image data.

5. A medical information processing device comprising processing circuitry configured to:
acquire first information regarding medical data and second information regarding an analysis application used to analyze the medical data;
acquire correctness or incorrectness of an analysis result using the analysis application selected from a plurality of analysis applications on the basis of the first information and the second information;
generate correction information for correcting construction conditions for constructing the medical data when the acquired analysis result is incorrect; and
provide the correction information.

6. The medical information processing device according to claim 5, wherein
the medical data includes medical image data, and
the first information includes supplementary information of the medical image data.

7. The medical information processing device according to claim 5, wherein the construction conditions include reconstruction conditions at the time of generating reconstructed medical image data by reconstructing original medical image data.

8. The medical information processing device according to claim 5, wherein
the second information includes an application activation rule for the analysis application, and
the processing circuitry is further configured to:
generate rule correction information for correcting the application activation rule upon determining that a possibility of failure of analysis of the medical data is high; and
provide the rule correction information.

9. The medical information processing device according to claim 8, wherein the processing circuitry is further configured generate the correction information on the basis of the first information and the second information generated in the past, and the construction conditions when an analysis result using the analysis application selected on the basis of the first information and the second information is correct.

10. A medical information processing method, using a computer, comprising:
acquiring first information regarding medical data and second information regarding an analysis application used to analyze the medical data; and
determining a possibility of failure of analysis of the medical data by an analysis application selected from a plurality of analysis applications on the basis of the first information and the second information.
